(19) United States
(12) Patent Application Publication
(10) Pub. No.: US 2025/0228775 A1
(43) Pub. Date: Jul. 17, 2025

(54) ETHANOLIC RESINIFERATOXIN CONCENTRATE

(71) Applicant: Gruenenthal GmbH, Aachen (DE)

(72) Inventor: Marc SCHILLER, Aachen (DE)

(73) Assignee: Gruenenthal GmbH, Aachen (DE)

(21) Appl. No.: 19/022,222

(22) Filed: Jan. 15, 2025

(30) Foreign Application Priority Data
Jan. 15, 2024 (EP) 24151807.5
Jan. 22, 2024 (EP) 24153125.0
Jan. 22, 2024 (EP) 24153126.8
May 13, 2024 (EP) 24175474.6

(51) Int. Cl. A61K 9/08; A61K 9/00; A61K 31/357; A61K 47/10; A61P 29/02

(52) U.S. Cl. CPC A61K 9/08; A61K 9/0019; A61K 31/357; A61K 47/10; A61P 29/02

(57) ABSTRACT

The invention relates to a liquid pharmaceutical concentrate comprising or essentially consisting of Resiniferatoxin in an amount of less than 3.20 μg, preferably at most 900 ng, more preferably at most 700 ng and ethanol. The composition according to the invention is particularly useful for the treatment of pain due to knee osteoarthritis. Prior to administration, the concentrate is diluted with a dilution liquid, preferably an aqueous dilution liquid, thereby obtaining a diluted composition, preferably a diluted solution. Said diluted composition or a partial volume thereof is subsequently administered as dose unit, preferably by intraarticular injection into the knee.

Priority is claimed of European Patent Application No. 24 151 807.5, filed Jan. 15, 2024; European Patent Application No. 24 153 125.0, filed Jan. 22, 2024; European Patent Application No. 24 153 126.8, filed Jan. 22, 2024; and European Patent Application No. 24 175 474.6, filed May 13, 2024, the disclosures of which patent applications are hereby incorporated herein by reference.

The invention relates to a liquid pharmaceutical concentrate comprising or essentially consisting of Resiniferatoxin in an amount of less than 3.20 μg, preferably at most 900 ng, more preferably at most 700 ng and ethanol. The composition according to the invention is particularly useful for the treatment of pain due to knee osteoarthritis. Prior to administration, the concentrate is diluted with a dilution liquid, preferably an aqueous dilution liquid, thereby obtaining a diluted composition, preferably a diluted solution. Said diluted composition or a partial volume thereof is subsequently administered as dose unit, preferably by intraarticular injection into the knee.

Resiniferatoxin (RTX) is a naturally occurring compound produced by the Moroccan cactus like succulent Euphorbia resinfera. Resiniferatoxin is known as a transient receptor potential cation channel subfamily V member 1 (TRPV1) agonist. RTX is an ultrapotent capsaicin analog with a unique spectrum of pharmacological actions. The therapeutic window of RTX is broad, allowing for the full desensitization of pain perception and neurogenic inflammation without causing unacceptable side effects. Intravesical RTX was shown to restore continence in a subset of patients with idiopathic and neurogenic detrusor overactivity. RTX can also ablate sensory neurons as a “molecular scalpel” to achieve permanent analgesia. This targeted (intrathecal or epidural) RTX therapy holds great promise in cancer pain management. Intraarticular RTX is undergoing clinical trials to treat moderate-to-severe knee pain in patients with osteoarthritis. Similar targeted approaches may be useful in the management of post-operative pain or pain associated with severe burn injuries (P. Campiglia et al., Int J Mol Sci 2023, 24(20), 15042).

Resiniferatoxin has been suggested for treating various types of pain and various other disorders and conditions by various routes of administration.
C. Sila et al., BJU Int. 2001 September; 88(4):355-60. doi: 10.1046/j.1464-410x.2001.02339.x. report that seven patients with detrusor hyperreflexia were treated intravesically with Resiniferatoxin dissolved in 10% ethanol in saline (50 nmol/L solution in two and 100 nmol/L in five).
H. Kuo, J Urol. 2003 September; 170(3):835-9. doi: 10.1097/01.ju.0000081652.31524.27 discloses intravesical Resiniferatoxin therapy with 10 ml of 100 nM Resiniferatoxin in 10% ethanol solution for 40 minutes.
M. de Sèze et al., J Urol. 2004 January; 171(1):251-5. doi: 10.1097/01.ju.0000100385.93801.d4 relates to a comparison of the efficacy and tolerability of nonalcohol capsaicin vs. Resiniferatoxin in 10% alcohol in neurogenic patients with detrusor hyperreflexia.
C. Silva et al., Eur Urol. 2005 October; 48(4):650-5. doi: 10.1016/j.eururo.2005.04.012 relates to an investigation wherein patients with spinal neurogenic detrusor overactivity were randomized to receive intravesically 50 nM Resiniferatoxin dissolved in 10% ethanol in saline.
E. Y. Kissin et al., Anesth Analg. 2005 November; 101(5):1433-1439. doi: 10.1213/01.ANE. 0000180998.29890.B0. relates to the effects of intraarticular Resiniferatoxin in experimental knee-joint arthritis.
J. C. Shin et al., Spinal Cord. 2006 May; 44(5):309-14. doi: 10.1038/sj.sc.3101851 relates to an investigation wherein 100 ml of Resiniferatoxin solution, at a concentration of 100 nM diluted in 10% ethanol, was intravesically instilled into the bladder of 15 spinal cord injury patients with neurogenic detrusor overactivity.
C. Silva et al., BMC Urol. 2007 June 11:7:9. doi: 10.1186/1471-2490-7-9 relates to an investigation wherein patients with overactive bladder were instilled with 100 ml of 50 nM Resiniferatoxin in 10% ethanol in saline.
I. Vetter et al., Eur J Pain. 2008 May; 12(4):441-54. doi: 10.1016/j.ejpain.2007.07.001 report that ethanol can sensitize TRPV1-mediated responses, but the pathways contributing to the potentiation of TRPV1 by ethanol have not been clearly defined. The presented results suggest that morphine may be of limited use in inhibiting nociceptive TRPV1 responses that have been sensitized by exposure to ethanol.
I. Kissin et al., Curr Top Med Chem. 2011; 11(17):2159-70. doi: 10.2174/156802611796904924 reviews therapeutic targeting of TRPV1 by Resiniferatoxin, from preclinical studies to clinical trials.
M. J. Iadarola et al., Resiniferatoxin for Pain Treatment: An Interventional Approach to Personalized Pain Medicine, Open Pain J. 2013; 6: 95-107 reviews preclinical and clinical studies related to Resiniferatoxin. Pain conditions that may be susceptible to treatment with local injection or topical Resiniferatoxin include osteoarthritis, whereas Resiniferatoxin is directly injected into the joint. No dosages are mentioned.
Y. Kim et al., The effects of intra-articular resiniferatoxin on monosodium iodoacetate-induced osteoarthritic pain in rats, Korean J Physiol Pharmacol 2016; 20(1):129-136 teaches that based upon preclinical studies in rats, local delivery of Resiniferatoxin may be beneficial to osteoarthritis patients. However, the major problem with Resiniferatoxin treatment is acute toxicity. No dosages for treating humans are mentioned.
H. Aizawa et al., Int J Urol. 2016 November; 23(11):952-956. doi: 10.1111/iju.13181 relates to an examination whether systemic Resiniferatoxin treatment can desensitize the single-unit afferent activities of Aδ- and C-fibers in rat primary bladder mechanosensitive afferent nerves. Resiniferatoxin (0.3 mg/kg) or its vehicle (10% ethanol) was injected subcutaneously to female Sprague-Dawley rats after the first eye-wipe behavior test with capsaicin.
M. J. Iadarola et al., Long-term pain relief in canine osteoarthritis by a single intra-articular injection of resiniferatoxin, a potent TRPV1 agonist, Pain. 2018 October; 159(10): 2105-2114 document a significant and prolonged analgesic effect of a single intraarticular injection of 10 μg of RTX in naturally-occurring canine osteoarthritis, which is physiologically, and symptomatically analogous to human osteoarthritis. 7 dogs with osteoarthritis were treated with a single intraarticular injection of 10 μg of Resiniferatoxin. Dose estimations, and other critical information from previous canine studies translated directly to the human cancer pain trial. This pilot study is said to provide proof-of-concept data for translation to a human osteoarthritis trial. No dosages for treating humans are quantified.
D. Leiman et al., abstract 197, Osteoarthritis and Cartilage 28 (2020) S86-S527 report about preliminary results from a phase 1b double-blind study to assess the safety, tolerability and efficacy of intraarticular Resiniferatoxin or placebo for the treatment of moderate to severe pain due to osteoarthritis of the knee. In this multicenter, randomized, double-blind, placebo-controlled study, eligible subjects aged 35-85 years, with baseline pain score in the index knee of ≥5 but 9 (WOMAC pain subscale question A1) were treated with a one-time intra-articular dose of Resiniferatoxin at escalating dose level cohorts of 5 μg, 12.5 μg, 20 μg and 30 μg in 5 or 10 mL of saline, or saline placebo. Resiniferatoxin demonstrated safety when given as onetime intraarticular injection up to the planned highest dose of 30 μg. Preliminary efficacy was also observed. Anecdotal long-term follow up suggests sustained pain relief beyond day 365.
A. Szallasi, Resiniferatoxin: Nature's Precision Medicine to Silence TRPV1-Positive Afferents, Int. J. Mol. Sci. 2023, 24, 15042 reviews medical utility of Resiniferatoxin. A breakthrough therapy designation for intraarticular Resiniferatoxin at a dose of 12.5 μg to treat pin associated with knee osteoarthritis is discussed (cf. s. Lopez et al., J. Analgesics of the Future: IA Resiniferatoxin for Osteoarthritic Knee Pain. Pract PainManag. 2022; 22 (2), updated Mar. 1, 2022).

US 2001 0006982 A1 (WO 1999/009970 A1) relates to a method of treating neurogenic urinary dysfunction that comprises contacting urinary bladder mucosa of a patient afflicted with neurogenic urinary dysfunction with an effective dose of a homovanilloid compound, in particular a compound selected from the group RTX, TYX, 20-homovanillyl-mezerein or 20-homovanillyl-12-deoxyphorbol-13-phenylacetate.

US 2004 0146590 A1, US 2010 0222385 A1, US 2013 0210905 A1, US 2015 0051271 A1, and US 2018 0117004 A1 provide methods and kits for the selective ablation of pain-sensing neurons. The methods comprise administration of a vanilloid receptor agonist to a ganglion in an amount that causes death of vanilloid receptor-bearing neurons.

US 2004 0161481 A1 (WO 2004/056305 A2) provides compositions and methods for relieving pain at a site in a human or animal in need thereof by administering at a discrete site in a human or animal in need thereof a dose of capsaicin in an amount effective to denervate a discrete site without eliciting an effect outside the discrete location, the dose of capsaicin ranging from 1 μg to 3000 μg.

US 2005 0019258 A1 (WO 2005/004883 A2) relates to a method for the evaluation of potential therapeutic agents for the treatment of incontinence, as well as to a method for discovering new therapeutic agents for the treatment of incontinence.

US 2005 0019436 A1 (WO 2004/058286 A1) and US 2007 0036876 A1 (WO 2004/056305 A2) disclose a method for attenuating pain at a site in a human or animal in need thereof, comprising: administering at a discrete painful site in a human or animal in need thereof a single injectable or implantable dose of a capsaicinoid in an amount effective to denervate said discrete site without eliciting an effect outside the discrete location and to attenuate pain emanating from said site, said effective dose being from about 1 μg to about 5000 μg of capsaicin or a therapeutically equivalent dose of a capsaicinoid other than capsaicin.

US 2006 0269628 A1 (WO 2004/058286 A1) provides compositions and methods for attenuating or relieving pain at a site in a human or animal in need thereof by infiltrating at a surgical site or open wound in a human or animal a dose of capsaicinoid in an amount effective to denervate the surgical site or open wound substantially without eliciting an effect outside the surgical site or open wound.

WO 2008/109026 A1 relates to compositions and methods for the treatment of neuropathic or neurogenic pain and/or disorders at least partially associated with neurogenic dysfunction comprising the use of a pharmaceutical composition comprising at least one homovanilloid compound and at least one permeation enhancer.

US 2008 0139641 A1 (WO 2006/069451 A1) relates to a formulation of Resiniferatoxin for the preparation of an agent for the treatment of different pain conditions.

US 2008 0260791 A1 (WO 2004/058286 A1) relates to an injectable or implantable pharmaceutical composition for attenuating pain at a site in a human or animal in need thereof, consisting essentially of a capsaicinoid selected from the group consisting of from 1 μg to 5000 μg of capsaicin, a therapeutically equivalent amount of one or more other capsaicinoids, and combinations thereof; in a pharmaceutically acceptable vehicle for injection or implantation.

US 2009 0209633 A1 (WO 2008/011532 A2) relates to a method of treating inflammatory pain conditions that involves administering an effective amount of a TRPV1 agonist, such as Resiniferatoxin, Tinyatoxin and related potent agonists and their analogs, to a patient to selectively induce nerve terminal depolarization block and/or nerve terminal death in select TRPV1-containing neurons, to provide the desired pain relief without significant permanent damage to cell bodies of the select TRPV-1 containing neurons.

US 2010 047181 A1 (WO 2006 066419 A 1) relates to a mixture comprising a vanilloid receptor agonist and a substance inhibiting nerve regeneration. Said mixture is suitable as a painkiller.

US 2010 0137344 A1 (WO 2008/015403 A1) relates to agents which are capable of inducing analgesia in chronic neuropathic pain, associated methods and uses thereof, in particular compounds capable of activating the TRPM8 receptor for the treatment of chronic neuropathic pain.

US 2014 0142073 A1 (WO 2014/075084A2) and US 2015 0133561 A1 (WO 2015/073577 A1) disclose capsaicinoid formulations and methods of treatment which can be utilized to treat/attenuate pain in mammals. Typically, administration is via injection at a discrete site to provide pain relief for an extended period of time. The formulations are administered in a pharmaceutically acceptable vehicle. The aqueous pharmaceutical compositions utilize pharmaceutically acceptable delivery vehicles that are single phase aqueous/water systems composed of pharmaceutically acceptable solvents including polyethylene glycol, ethanol and a non-ionic surfactant, e.g. polysorbate 80 (PS 80); buffers; sodium chloride and/or sugar to control osmotic pressure gradients; and hyaluronic acid to control the viscosity of the formulation and aid in formulation stability.

US 2014 0187619 A1 (WO 2013/012892 A2) relates to methods of treatment in subjects suffering from diabetes mellitus or obesity. The methods comprise the step of administering an active agent directly to the small intestine in the subject. The active agents include analgesic agents and, in particular, antinociceptive agents such as capsaicin, Resiniferatoxin, and their analogues.

US 2015 0080460 A1 relates to a method for administration of a formulation of Resiniferatoxin (RTX) directly onto the epicardium, comprising applying a formulation of RTX directly onto the epicardium under the pericardial sac.

US 2015 0190509 A1 (WO 2014/019095 A1) relates to a Resiniferatoxin solution having an enhanced storage stability, in which the Resiniferatoxin is dissolved in a body-compatible solvent which contains a protective gas in solution, wherein the amount of the protective gas is at least 1 wt.-% of the saturation amount of the protective gas in the solvent at ambient temperature and normal pressure. The solution can be used in particular for the treatment of intra-articular pain.

US 2019 0076396 A1 (WO 2019/049112 A1) relates to formulations of Resiniferatoxin (RTX) for intrathecal, intraganglionic intraarticular and pericardial administration, more specifically alcohol-free formulations of RTX comprising a solubilizing component, a monosaccharide or sugar alcohol, a saline buffer, and RTX, and having narrow ranges for pH range and specific gravity. It is alleged that for intraarticular administration, a typical volume injected into an adult knee would be from 3 ml to 10 ml, delivering a total amount of RTX from 5 ng to 50 μg, and that often the amount administered would be from 200 ng to 10 μg.

US 2019 0321493 A1 (WO 2018/094262 A2) provides materials and methods for treating regional pain. For example, compositions including one or more analgesics can be selectively administered (e.g., by image-guided injection) to one or more nerves to treat a mammal having regional pain.

WO 2020 132553A1 discloses methods of administering resiniferatoxin (RTX) perineurally for treatment of maladaptive pain, and compositions for use in such methods.

US 2020 0261409 A1 (WO 2017/087803 A1) discloses methods for treating pain using Resiniferatoxin. The Resiniferatoxin may be administered to certain anatomic sites using image-guided delivery. The Resiniferatoxin may be administered in low doses in the range of 0.5 to 3.0 μg of Resiniferatoxin in a human patient.

US 2020 0323800 A1 (WO 2015/160941 A1) relates to TRPV1 selective agonist compositions including a capsaicinoid, a surfactant and an extended release agent, and to methods of manufacture and methods of providing pain relief as well as treating a variety of disorders with such compositions.

US 2021 0007998 A1 relates to nanoparticles comprising Resiniferatoxin (RTX) encapsulated in a poly(lactic-co-glycolic acid) (PLGA) polymer and compositions, especially topical compositions, comprising the nanoparticles.

US 2021 0299090 A 1 (WO 2020 139797A1) discloses a method for treating Parkinson's Disease (PD) comprising administering an effective amount of Resiniferatoxin (RTX) by an intrathecal or intracisternal administration.

US 2021 0393515 A1 relates to methods of administering Resiniferatoxin (RTX) perineurally for treatment of maladaptive pain, and compositions for use in such methods.

US 2022 0008384 A1 (WO 2020/191506 A1) relates to a composition comprising capsaicin or a capsaicinoid is used in a method for postoperative pain control. The composition is administered to a site intended for surgery in a patient at least one day before surgery is actually performed.

US 2022 0096428 A1 (WO 2020/154261 A1) relates to methods of administering Resiniferatoxin (RTX) for treatment of osteoarthritis (OA) pain, and compositions for use in such methods.

US 2023 0051321 A1 (WO 2021/209450 A1) relates to non-aqueous solid and liquid compositions comprising Resiniferatoxin and a surfactant. The non-aqueous solid and liquid compositions may be used to prepare aqueous compositions that are used in the treatment of pain, specifically osteoarthritis-related joint pain.

US 2023 0143545 A1 (WO 2021/202084 A1) relates to a method for treating pulmonary inflammatory disease comprising administering an effective amount of Resiniferatoxin (RTX) by an epidural, peri-ganglionic or intra-ganglionic administration. In some embodiments, the dose of RTX for an adult human is from about 0.1 μg to about 100 μg.

US 2023 0270713 A1 (WO 2021/257956 A1) relates to methods of administering Resiniferatoxin (RTX) intravesically for treatment of bladder pain, and compositions for use in such methods.

Further, Resiniferatoxin has been suggested for treating joint pain such as pain due to osteoarthritis of the knee. While it is alleged in US 2019 0076396 A1 (WO 2019/049112 A1) that for intraarticular administration, a typical volume injected into an adult knee would be from 3 ml to 10 ml, delivering a total amount of RTX from 5 ng to 50 μg, and that often the amount administered would be from 200 ng to 10 μg, the clinically tested doses of Resiniferatoxin in fact were in the μg scale, typically 3 μg, 6 μg, 12.5 μg, 20 μg, or 30 μg.

A completed clinical trial (www.clinicaltrials.gov, identifier NCT03542838) relates to a study of Resiniferatoxin for knee pain in moderate to severe osteoarthritis. According to the study plan, Resiniferatoxin was administered as a one-time dose, intraarticularly at a dose level of 5 μg, 12.5 μg, 20 μg, 25 μg, or 30 μg. The study was completed on Nov. 2, 2021 and no results have been posted.

A withdrawn clinical trial (www.clinicaltrials.gov, identifier NCT04044742) was planned as a phase 3 study to evaluate the efficacy and safety of Resiniferatoxin for pain due to osteoarthritis of the knee. According to the study plan, 12.5 μg of Resiniferatoxin in 5 ml volume should be administered intraarticularly as a one-time dose.

A withdrawn clinical trial (www.clinicaltrials.gov, identifier NCT04386980) was planned to evaluate resiniferatoxin in patients with knee osteoarthritis whose total knee replacement surgery is delayed. According to the study plan, 12.5 μg of Resiniferatoxin in 5 ml volume should be administered once intraarticularly.

An active, not recruiting clinical trial (www.clinicaltrials.gov, identifier NCT04885972) is planned as a study to evaluate intraarticular Resiniferatoxin to treat moderate to severe pain from knee osteoarthritis. According to the study plan, 7.5 μg, 10 μg, 12.5 μg, 15 μg, or 20 μg in 5 ml should be injected once intraarticularly.

With press release of Sep. 7, 2023, Sorrento Therapeutics announced positive phase 2a clinical trial results for Resiniferatoxin for the treatment of knee pain in moderate to severe osteoarthritis of the knee patients. The administered doses of Resiniferatoxin amounted to 7.5 to 20 μg.

The known methods and medicaments for treating knee joint pain, especially osteoarthritic knee joint pain, are not satisfactory in every respect and there is a demand for improved methods and medicaments.

Only a few pharmaceutical compositions containing Resiniferatoxin are known from the prior art.

The pharmaceutical compositions containing Resiniferatoxin that are known from the prior art are not satisfactory in every respect and there is a demand for improved pharmaceutical compositions. Especially when the administered dose of Resiniferatoxin is very low, e.g. about 400 ng, it is particularly challenging to provide satisfactory storage stability and shelf-life.

It is an object of the invention to provide pharmaceutical compositions containing Resiniferatoxin that have advantages compared to the pharmaceutical compositions of the prior art. The pharmaceutical compositions should provide satisfactory storage stability and shelf-life, should be easy to apply and administer, and should be producible on large scale in an economic manner. Further, the pharmaceutical compositions should be suitable for treating knee joint pain, especially osteoarthritic knee joint pain, should be safe and should have as little treatment emergent adverse events as possible.

These objects have been achieved by the subject-matter of the patent claims.

It has been surprisingly found that liquid pharmaceutical concentrates containing comparatively low doses of Resiniferatoxin have advantages when they are stored in ethanolic solution. Shortly prior to administration, the concentrates are diluted with a dilution liquid, preferably an aqueous dilution liquid. The thus obtained diluted compositions, preferably diluted solutions, or a partial volume thereof can then be administered as dose unit by injection.

Experimental data indicate that such ethanolic concentrates exhibit improved storage stability especially when they are stored in specific glass vials. Storage stability is not only satisfactory when being stored at low temperature (e.g. −18° C. or 5° C.) and even at room temperature (25° C.). Theses advantages are particularly relevant when the amount of Resiniferatoxin per ethanolic concentrate is less than 3.20 μg, preferably at most 900 ng, more preferably at most 700 ng, e.g. about 0.68 μg (680 ng), or e.g. about 0.46 μg (460 ng). As a consequence of the achieved stability even at room temperature, the supply chain is less complex.

Further, it has been surprisingly found that Resiniferatoxin provides relief of knee joint pain, especially osteoarthritic knee joint pain, when it is intraarticularly administered at significantly lower doses than suggested in the prior art. While it has been suggested in the prior art to treat osteoarthritic knee joint pain by intraarticularly administering doses of Resiniferatoxin on the μg scale, e.g. 12.5 μg, it has now been surprisingly found that a much lower dose of Resiniferatoxin amounting to only 400 ng or even only 100 ng provides satisfactory analgesia, especially when the administered dose unit contains ethanol in aqueous media (for the purpose of the specification also referred to as “aqueous ethanol”). It can be expected that such lower doses provide better tolerability and safety than the conventional doses of Resiniferatoxin on the μg scale.

Still further, the ethanol content in the dose unit that is administered to the patient unexpectedly has pharmacological advantages, especially with respect to tissue distribution being significantly enhanced.

Furthermore, it has been surprisingly found that even such ultra low doses of Resiniferatoxin provide prolonged pain relief of several months after intraarticular administration. Thus, the ultra low doses according to the invention appear particularly useful for long term treatment wherein the patient receives repeated intraarticular administration over several months or years.

### SUMMARY OF THE INVENTION

A first aspect of the invention relates to a liquid pharmaceutical concentrate comprising or essentially consisting of
Resiniferatoxin in an amount of less than 3.20 μg, preferably at most 900 ng, more preferably at most 700 ng; and
ethanol.

### BRIEF DESCRIPTION OF THE DRAWING

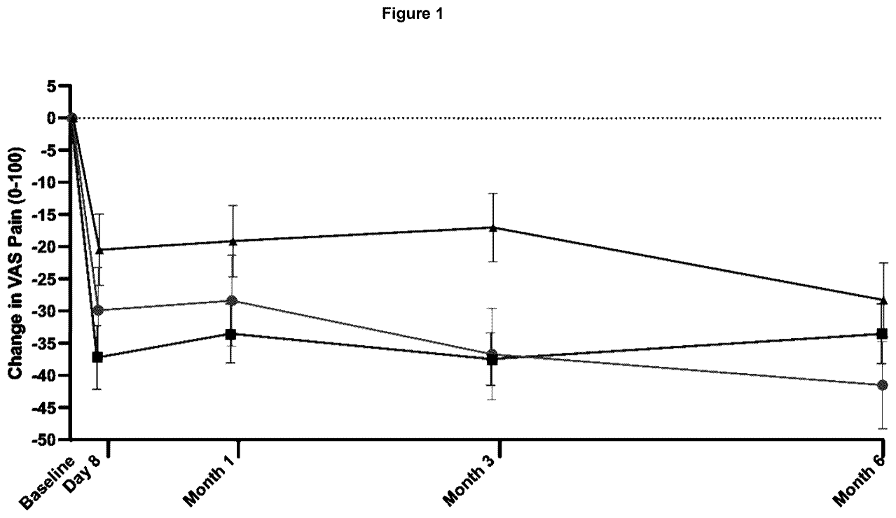

The invention will now be described in greater detail with reference to the drawing, wherein FIG. 1 is a graph that shows the efficacy in terms of visual analog scale pain in motion over time.

For the purpose of the specification, it is distinguished between
a “liquid pharmaceutical concentrate” (also interchangeably referred to as “concentrate” or “ethanolic concentrate”);
a “dilution liquid”, preferably “aqueous dilution liquid”;
a “diluted composition”, preferably “diluted solution”, which is obtained when the liquid pharmaceutical concentrate and the dilution liquid are combined (e.g. mixed) with one another; and
a “dose unit” (also interchangeably referred to as “administered dose unit”), which may either correspond to the diluted composition as such, or which may be a partial volume of the diluted composition.

The liquid pharmaceutical concentrate is for the purpose of storing low amounts of Resiniferatoxin at satisfactory storage stability and shelf-life. Preferably, every unit of liquid pharmaceutical concentrate containing Resiniferatoxin in an amount of less than 3.20 μg, preferably at most 900 ng, more preferably at most 700 ng is for a single administration. Preferably, the liquid pharmaceutical concentrate is contained in a specific glass vial that contributes to the improved storage stability and shelf-life so that it is even possible to store the liquid pharmaceutical concentrate at room temperature (25° C.). Preferably, every glass vial contains medication for a single administration (single dose unit).

The dilution liquid can be freshly prepared or stored in separate form, i.e. in a separate container. The dilution liquid preferably contains all those ingredients and excipients, which should be administered together with the Resiniferatoxin but which should not be contained in the liquid pharmaceutical concentrate during storage thereof, as they might e.g. have a negative impact on storage stability and shelf-life.

The diluted composition is prepared shortly prior to administration, preferably by adding the dilution liquid to the liquid pharmaceutical concentrate. For that purpose, the liquid pharmaceutical concentrate is preferably contained in a container, e.g. glass vial, having a sufficient fill capacity such that it cannot only contain the liquid pharmaceutical concentrate but additionally receive a predetermined volume of the dilution liquid. The diluted composition should ensure that a well defined dose of Resiniferatoxin may be taken, e.g. drawn up into a syringe, and administered as dose unit, preferably by intraarticular injection. Preferably, every dose unit of diluted composition containing Resiniferatoxin in an amount of less than 3.20 μg, preferably at most 900 ng, more preferably at most 700 ng is for a single administration.

As it will typically be difficult to completely withdraw the total volume of the diluted composition from the container, e.g. glass vial, the volume of the diluted composition (and its corresponding content of Resiniferatoxin) preferably exceeds the volume of the dose unit for administration (and its corresponding administered dose of Resiniferatoxin). In other words, the volume of the administered dose unit preferably does not correspond to the total volume of the diluted composition that is obtained by combining the liquid pharmaceutical concentrate and the dilution liquid with one another. Rather, the volume of the administered dose unit is preferably smaller than the total volume of the diluted composition. This facilitates withdrawal of the right dose of Resiniferatoxin from the container and subsequent administration thereof.

The liquid pharmaceutical concentrate, the diluted composition, and the dose unit preferably contain medication for a single administration, preferably by injection (single dose unit). It is contemplated that the amount of Resiniferatoxin contained in the liquid pharmaceutical concentrate and in the diluted composition may exceed the dose of Resiniferatoxin that is finally administered. Nonetheless, neither the liquid pharmaceutical concentrate nor the diluted composition are preferably multiple dose units. On the contrary, when only a partial volume of the diluted composition is administered as a single dose, the excessive Resiniferatoxin remaining in the residual diluted composition is preferably disposed.

It is contemplated, however, that the liquid pharmaceutical concentrate is provided for multiple treatments, i.e. after dilution contains more than a single dose unit for administration. For example, when the liquid pharmaceutical concentrate is provided for treating 4 subjects, it preferably contains Resiniferatoxin in an amount of 4 times 670 ng, i.e. 2.68 μg. The dilution liquid preferably has a total volume of 4 times 8 ml, i.e. 32 ml. Preferably, aliquots of 5 ml thus each containing about 400 ng Resiniferatoxin are drawn as a dose unit into a syringe and injected into the knee of the patients.

In each case, said dose unit preferably
contains Resiniferatoxin at a dose within the range of 400±200 ng, preferably 400±175 ng, more preferably 400±150 ng, still more preferably 400±125 ng, yet more preferably 400±100 ng, even more preferably 400±75 ng, most preferably 400±50 ng, and in particular 400±25 ng;
contains ethanol at a volume concentration within the range of 5.0±4.0 vol.-%; preferably 5.0±3.5 vol.-%, more preferably 5.0±3.0 vol.-%, still more preferably 5.0±2.5 vol.-%, yet more preferably 5.0±2.0 vol.-%, even more preferably 5.0±1.5 vol.-%, most preferably 5.0±1.0 vol.-%, and in particular 5.0±0.5 vol.-%, relative to the total volume of the dose unit; and/or
has a total volume within the range of 5.0±4.0 ml, preferably 5.0±3.5 ml, more preferably 5.0±3.0 ml, still more preferably 5.0±2.5 ml, yet more preferably 5.0±2.0 ml, even more preferably 5.0±1.5 ml, most preferably 5.0±1.0 ml, and in particular 5.0±0.5 ml.

Two particularly preferred embodiments of the invention are compiled in the table here below:

| | | #1 | | #2 | |
|---|---|---|---|---|---|
| liquid pharmaceutical | volume | 0.421 | ml | 0.289 | ml |
| concentrate | weight | 0.332 | g | 0.228 | g |
| | RTX concentration | 1.6 μg RTX | | 1.6 μg RTX | |
| | | per ml | | per ml | |
| dilution liquid | volume | 8.0 | ml | 5.5 | ml |
| diluted composition | volume | ~8.4 | ml | ~5.8 | ml |

| | | | |
|---|---|---|---|
| dose unit | administered dose | 400 ng RTX | 400 ng RTX |
| | of RTX in | in 5 ml | in 5 ml |
| | administered | | |
| | volume | | |

According to embodiment #1, the diluted composition (i.e. after reconstitution) has a total volume of more than 8.0 ml such that after withdrawal of the dose unit having a volume of 5.0 ml, a large amount of the diluted composition remains in the vial and is to be discarded. According to embodiment #2, the diluted composition (i.e. after reconstitution) has a total volume of less than 6.0 ml such that after withdrawal of the identical dose unit likewise having a volume of 5.0 ml, a much lower amount of the diluted composition remains in the vial and is to be discarded.

Ethanol is preferably contained in the liquid pharmaceutical concentrate, but typically not in the dilution liquid. The ethanol has different advantages. On the one hand, ethanol provides storage stability to the Resiniferatoxin that is contained in the liquid pharmaceutical concentrate. On the other hand, ethanol improves distribution in the tissue after injection of the dose unit and thus also has a pharmacological effect.

The invention combines the advantages of administering Resiniferatoxin at very low dosages with the advantages of ethanol.

For the purpose of the specification, “essentially consisting of” means that specific further components can be present, namely those not materially affecting the essential characteristics of the mixture. Preferably, the total content of such further components relative to the total weight of the mixture is at most 1.0 wt.-%, more preferably at most 0.5 wt.-%, still more preferably at most 0.1 wt.-%, most preferably at most 0.001 wt.-%, and in particular below the analytical detection limit.

### DETAILED DESCRIPTION OF THE INVENTION

Resiniferatoxin (RTX, C₃₇H₄₀O₉, Mr 628.718 g/mol, CAS 57444-62-9, DrugBank Accession No DB06515) is the compound of the following structure:

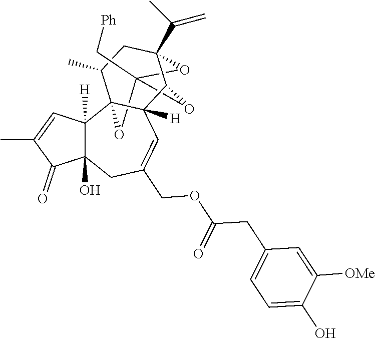 [SMILES: C=C(C)[C@]12C[C@@H](C)[C@@]34OC(Cc5ccccc5)(O[C@@H]1[C@@H]3C=C(COC(=O)Cc1ccc(O)c(OC)c1)C[C@]1(O)C(=O)C(C)=C[C@@H]41)O2]

and has the systematic name 4-hydroxy-3-methoxy-benzeneacetic acid, [(2S,3aR,3bS,6aR,9aR,9bR, 10R,11aR)-3a,3b,6,6a,9a,10,11,11a-octahydro-6a-hydroxy-8,10-dimethyl-11a-(1-methylethenyl)-7-oxo-2-(phenylmethyl)-7H-2,9b-epoxyazuleno[5,4-e]-1,3-benzodioxol-5-yl]methyl ester. It is commercially available.

Preferably, the concentrate according to the invention contains Resiniferatoxin in an amount of at most 3.00 μg, preferably at most 2.75 μg, more preferably at most 2.50 μg, still more preferably at most 2.25 μg, yet more preferably at most 2.00 μg, even more preferably at most 1.75 μg, most preferably at most 1.50 μg, and in particular at most 1.25 μg.

Preferably, the concentrate according to the invention contains Resiniferatoxin in an amount of at most 1000 ng, preferably at most 950 ng, more preferably at most 900 ng, still more preferably at most 850 ng, yet more preferably at most 800 ng, even more preferably at most 750 ng, most preferably at most 700 ng, and in particular at most 680 ng.

In preferred embodiments, the concentrate according to the invention contains Resiniferatoxin in an amount within the range of 460±200 ng, preferably 460±175 ng, more preferably 460±150 ng, still more preferably 460±125 ng, yet more preferably 460±100 ng, even more preferably 460±75 ng, most preferably 460±50 ng, and in particular 460±25 ng.

In other preferred embodiments, the concentrate according to the invention contains Resiniferatoxin in an amount within the range of 670±200 ng, preferably 670±175 ng, more preferably 670±150 ng, still more preferably 670±125 ng, yet more preferably 670±100 ng, even more preferably 670±75 ng, most preferably 670±50 ng, and in particular 670±25 ng.

Preferably, the concentrate according to the invention contains Resiniferatoxin at a concentration of at least 0.20 μg·ml⁻¹, preferably at least 0.40 μg·ml⁻¹, more preferably at least 0.60 μg·ml⁻¹, still more preferably at least 0.80 μg·ml⁻¹, yet more preferably at least 1.00 μg·ml⁻¹, even more preferably at least 1.20 μg·ml⁻¹, most preferably at least 1.40 μg·ml⁻¹, and in particular at least 1.50 μg·ml⁻¹.

Preferably, the concentrate according to the invention contains Resiniferatoxin at a concentration of at most 7.00 μg·ml⁻¹, preferably at most 6.50 μg·ml⁻¹, more preferably at most 6.00 μg·ml⁻¹, still more preferably at most 5.50 μg·ml⁻¹, yet more preferably at most 5.00 μg·ml⁻¹, even more preferably at most 4.50 μg·ml⁻¹, most preferably at most 4.00 μg·ml⁻¹, and in particular at most 3.50 μg·ml⁻¹.

Preferably, the concentrate according to the invention contains Resiniferatoxin at a concentration of at most 3.20 μg·ml⁻¹, preferably at most 3.00 μg·ml⁻¹, more preferably at most 2.80 μg·ml⁻¹, still more preferably at most 2.60 μg·ml⁻¹, yet more preferably at most 2.40 μg·ml⁻¹, even more preferably at most 2.20 μg·ml⁻¹, most preferably at most 2.00 μg·ml⁻¹, and in particular at most 1.80 μg·ml⁻¹.

Preferably, the concentrate according to the invention contains Resiniferatoxin at a concentration within the range of 1.60±1.50 μg·ml⁻¹, preferably 1.60±1.40 μg·ml⁻¹, more preferably 1.60±1.20 μg·ml⁻¹, still more preferably 1.60±1.00 μg·ml⁻¹, yet more preferably 1.60±0.80 μg·ml⁻¹, even more preferably 1.60±0.60 μg·ml⁻¹, most preferably 1.60±0.40 μg·ml⁻¹, and in particular 1.60±0.20 μg·ml⁻¹.

Preferably, the concentrate according to the invention contains Resiniferatoxin at a concentration of more than 1.0 μmol·l⁻¹, preferably at least 1.2 μmol·l⁻¹, more preferably at least 1.4 μmol·l⁻¹, still more preferably at least 1.6 μmol·l⁻¹, yet more preferably at least 1.8 μmol·l⁻¹, even more preferably at least 2.0 μmol·l⁻¹, most preferably at least 2.2 μmol·l⁻¹, and in particular at least 2.4 μmol·l⁻¹.

Preferably, the concentrate according to the invention contains Resiniferatoxin at a concentration of less than 10 μmol·l⁻¹, preferably at most 9.0 μmol·l⁻¹, more preferably at most 8.0 μmol·l⁻¹, still more preferably at most 7.0 μmol·l⁻¹, yet more preferably at most 6.0 μmol·l⁻¹, even more preferably at most 5.0 μmol·l⁻¹, most preferably at most 4.0 μmol·l⁻¹, and in particular at most 3.0 μmol·l⁻¹.

Preferably, the concentrate according to the invention contains Resiniferatoxin at a concentration within the range of 2.5±2.4 μmol·l⁻¹, preferably 2.5±2.1 μmol·l⁻¹, more preferably 2.5±1.8 μmol·l⁻¹, still more preferably 2.5±1.5 μmol·l⁻¹, yet more preferably 2.5±1.2 μmol·l⁻¹, even more preferably 2.5±0.9 μmol·l⁻¹, most preferably 2.5±0.6 μmol·l⁻¹, and in particular 2.5±0.3 μmol·l⁻¹.

Preferably, the concentrate according to the invention has a total volume of at least 50 μl, preferably at least 100 μl, more preferably at least 150 μl, still more preferably at least 200 μl, yet more preferably at least 250 μl, even more preferably at least 300 μl, most preferably at least 350 μl, and in particular at least 400 μl.

Preferably, the concentrate according to the invention has a total volume of at most 800 μl, preferably at most 750 μl, more preferably at most 700 μl, still more preferably at most 650 μl, yet more preferably at most 600 μl, even more preferably at most 550 μl, most preferably at most 500 μl, and in particular at most 450 μl.

In preferred embodiments, the concentrate according to the invention has a total volume within the range of 290±200 μl, preferably 290±175 μl, more preferably 290±150 μl, still more preferably 290±125 μl, yet more preferably 290±100 μl, even more preferably 290±75 μl, most preferably 290±50 μl, and in particular 290±25 μl.

In other preferred embodiments, the concentrate according to the invention has a total volume within the range of 420±200 μl, preferably 420±175 μl, more preferably 420±150 μl, still more preferably 420±125 μl, yet more preferably 420±100 μl, even more preferably 420±75 μl, most preferably 420±50 μl, and in particular 420±25 μl.

Preferably, the concentrate according to the invention is non-aqueous.

Preferably, the concentrate according to the invention does not contain a buffer.

Preferably, the concentrate according to the invention does not contain a surfactant.

Preferably, the concentrate according to the invention does not contain isotonizing agents (isotonic agents, tonicity agents).

Preferably, the concentrate according to the invention essentially consists of Resiniferatoxin and ethanol.

Another aspect of the invention relates to the concentrate according to the invention as described above for use in the treatment of pain, preferably joint pain, more preferably knee joint pain, preferably after dilution of the concentrate with a dilution liquid.

Another aspect of the invention relates to a method of treating pain, preferably joint pain, more preferably knee joint pain, the method comprising the step of administering to a subject in need thereof the concentrate according to the invention as described above, preferably after dilution of the concentrate with a dilution liquid.

Another aspect of the invention relates to the use of Resiniferatoxin for the manufacture of the concentrate according to the invention as described above for treating pain, preferably joint pain, more preferably knee joint pain, preferably after dilution of the concentrate with a dilution liquid.

Preferably, the pain is associated with osteoarthritis, preferably knee osteoarthritis.

Unless expressly stated otherwise, the osteoarthritis of the knee is in accordance with the International Classification of Diseases, ICD-11 (version 1/2023), FAO1.

Osteoarthritis of the knee is described therein as primary osteoarthritis occurring in an otherwise intact knee joint, involving genetically related, age-related or use-related degeneration with microscopic and macroscopic anatomical changes, which ultimately limit motion in one or more joints. Changes to the joint include increasing cartilage loss and osseous transformation such as sclerosis, osteophyte formation and cysts as well as potential inflammatory changes in surrounding soft tissue structures.

In preferred embodiments, the osteoarthritis of the knee is bilateral (XK9J), left (XK8G), right (XK9K), or unilateral unspecified (XK70).

In preferred embodiments, the pain that is associated to the osteoarthritis of the knee, i.e. the osteoarthritic knee joint pain, is selected from chronic post traumatic pain (MG30.20) and chronic secondary musculoskeletal pain associated with structural changes (MG30.31).

Within chronic post traumatic pain (MG30.20) or chronic secondary musculoskeletal pain associated with structural changes (MG30.31), the pain may have severity selected from mild pain (XS5D), moderate pain (XS9Q), and severe pain (XS2E). Within chronic post traumatic pain (MG30.20) or chronic secondary musculoskeletal pain associated with structural changes (MG30.31), the pain may have alternative severity 1 selected from mild distress (XS3R), moderate distress (XS7C), and severe distress (XS7N). Within chronic post traumatic pain (MG30.20) or chronic secondary musculoskeletal pain associated with structural changes (MG30.31), the pain may have alternative severity 2 selected from mild pain-related interference (XS5R), moderate pain-related interference (XS2L), and severe pain-related interference (XS2U). Within chronic post traumatic pain (MG30.20) or chronic secondary musculoskeletal pain associated with structural changes (MG30.31), the pain may have temporal pattern and onset selected from intermittent (XT5G), persistent (XT6Z), and persistent with overlaid attacks (XT5T).

Preferably, the pain is moderate to severe.

In preferred embodiments, the knee joint pain is osteoarthritic knee joint pain associated with osteoarthritis of Kellgren-Lawrence Grade 2 (minimal).

In preferred embodiments, the knee joint pain is osteoarthritic knee joint pain associated with osteoarthritis of Kellgren-Lawrence Grade 3 (moderate).

In preferred embodiments, the knee joint pain is osteoarthritic knee joint pain associated with osteoarthritis of Kellgren-Lawrence Grade 4 (severe).

In preferred embodiments, the knee joint pain is osteoarthritic knee joint pain associated with osteoarthritis of Ahlbäck Grade 1 (joint space<3 mm).

In preferred embodiments, the knee joint pain is osteoarthritic knee joint pain associated with osteoarthritis of Ahlbäck Grade 2 (joint space obliteration).

In preferred embodiments, the knee joint pain is osteoarthritic knee joint pain associated with osteoarthritis of Ahlbäck Grade 3 (minor bone attrition, 0-5 mm).

In preferred embodiments, the knee joint pain is osteoarthritic knee joint pain associated with osteoarthritis of Ahlbäck Grade 4 (moderate bone attrition. >5-10 mm).

In preferred embodiments, the knee joint pain is osteoarthritic knee joint pain associated with osteoarthritis of Ahlbäck Grade 5 (severe bone attrition, >10 mm).

The Ahlbäck and the Kellgren-Lawrence grading systems are known to the skilled person (S Ahlbäck, Osteoarthrosis of the knee: a radiographic investigation. Acta Radiol Suppl 1968, 277:7-72; J H Kellgren, J S Lawrence, Radiologic assessment of osteoarthritis. Ann Rheum Dis 1957 16:494-501) and can be compared to one another as follows:

| Ahlbäck | | Kellgren-Lawrence | |
|---|---|---|---|
| grade | definition | grade | definition |
| — | — | 1 | doubtful; minute osteophytes, doubtful |
| | | | significance |
| — | — | 2 | minimal; definite osteophytes, unimpaired |
| | | | joint space |
| 1 | joint space narrowing (joint space <3 | 3 | moderate; moderate diminution of joint |
| | mm) | | space |
| 2 | joint space obliteration | 4 | severe; joint space greatly of impaired |
| | | | with sclerosis subchondral bone |
| 3 | minor bone attrition (0-5 mm) | 4 | as above |
| 4 | moderate bone attrition (>5-10 mm) | 4 | as above |
| 5 | severe bone attrition (<10 mm) | 4 | as above |

Preferably, the pain is chronic.

Preferably, prior to administration, the concentrate is diluted with a dilution liquid, preferably an aqueous dilution liquid, thereby obtaining a diluted composition, preferably a diluted solution.

Preferably, the thus obtained diluted composition, either its total volume or a partial volume thereof, is devoted for subsequent administration.

Preferably, the dilution liquid is aqueous.

Preferably, the dilution liquid contains a surfactant, preferably a nonionic surfactant.

Preferably, the surfactant is D-α-tocopherol polyethylene glycol succinate (TPGS).

Preferably, the surfactant is selected from TPGS 200, TPGS 238, TPGS 400, TPGS 600, TPGS 1000, TPGS 2000, TPGS 3400, TPGS 3500, TPGS 4000, TPGS 6000, and mixtures thereof.

Preferably, the surfactant is D-a-tocopherol polyethylene glycol 1000 succinate (TPGS 1000).

Preferably, the dilution liquid is buffered.

Preferably, the dilution liquid has a pH value within the range of from 7.5 to 8.2.

Preferably, the dilution liquid has a pH value within the range of 8.0±0.8, preferably 8.0±0.7, more preferably 8.0±0.6, still more preferably 8.0±0.5, yet more preferably 8.0±0.4, even more preferably 8.0±0.3, most preferably 8.0±0.2, and in particular preferably 8.0±0.1.

Suitable buffers systems are known to the skilled person. Preferably, the buffer system is based upon trometamol (tris(hydroxymethyl)aminomethane, tromethamine, TRIS).

Preferably, the dilution liquid contains one or more isotonizing agents; preferably selected from sodium chloride, potassium chloride, calcium chloride, and combinations thereof.

Preferably, the dilution liquid has a total volume of at least 4.5 ml, more preferably at least 5.0 ml, still more preferably at least 5.5 ml, yet more preferably at least 6.0 ml, even more preferably at least 6.5 ml, most preferably at least 7.0 ml, and in particular at least 7.5 ml.

Preferably, the dilution liquid has a total volume of at most 12.0 ml, preferably at most 11.5 ml, more preferably at most 11.0 ml, still more preferably at most 10.5 ml, yet more preferably at most 10.0 ml, even more preferably at most 9.5 ml, most preferably at most 9.0 ml, and in particular at most 8.5 ml.

In preferred embodiments, the dilution liquid has a total volume within the range of 5.5±4.0 ml, preferably 5.5±3.5 ml, more preferably 5.5±3.0 ml, still more preferably 5.5±2.5 ml, yet more preferably 5.5±2.0 ml, even more preferably 5.5±1.5 ml, most preferably 5.5±1.0 ml, and in particular 5.5±0.5 ml.

In other preferred embodiments, the dilution liquid has a total volume within the range of 8.0±4.0 ml, preferably 8.0±3.5 ml, more preferably 8.0±3.0 ml, still more preferably 8.0±2.5 ml, yet more preferably 8.0±2.0 ml, even more preferably 8.0±1.5 ml, most preferably 8.0±1.0 ml, and in particular 8.0±0.5 ml.

Preferably, the diluted composition has a total volume of at least 5.0 ml, more preferably at least 5.5 ml, still more preferably at least 6.0 ml, yet more preferably at least 6.5 ml, even more preferably at least 7.0 ml, most preferably at least 7.5 ml, and in particular at least 8.0 ml.

Preferably, the diluted composition has a total volume of at most 12.5 ml, preferably at most 12.0 ml, more preferably at most 11.5 ml, still more preferably at most 11.0 ml, yet more preferably at most 10.5 ml, even more preferably at most 10.0 ml, most preferably at most 9.5 ml, and in particular at most 9.0 ml.

In preferred embodiments, the diluted composition has a total volume within the range of 5.8±4.0 ml, preferably 5.8±3.5 ml, more preferably 5.8±3.0 ml, still more preferably 5.8±2.5 ml, yet more preferably 5.8±2.0 ml, even more preferably 5.8±1.5 ml, most preferably 5.8±1.0 ml, and in particular 5.8±0.5 ml.

In other preferred embodiments, the diluted composition has a total volume within the range of 8.5±4.0 ml, preferably 8.5±3.5 ml, more preferably 8.5±3.0 ml, still more preferably 8.5±2.5 ml, yet more preferably 8.5±2.0 ml, even more preferably 8.5±1.5 ml, most preferably 8.5±1.0 ml, and in particular 8.5±0.5 ml.

Preferably, the diluted composition and thus also the administered dose unit has a pH value within the range of from 7.5 to 8.2.

In preferred embodiments, the diluted composition or a partial volume thereof is administered as dose unit.

When the total volume of the diluted composition is administered, the administered dose corresponds to the total amount of Resiniferatoxin that is contained in the diluted composition and that was originally contained in the concentrate before the dilution liquid was added, respectively.

When a partial volume of the diluted composition is administered, the administered dose is smaller than the total amount of Resiniferatoxin that is contained in the diluted composition and that was originally contained in the concentrate before the dilution liquid was added, respectively.

Preferably, the administered dose unit is administered by injection, preferably by intraarticular injection, more preferably into the knee.

For intraarticular injection the patient is preferably placed in the supine position. A small pillow or towel is preferably placed under the knee thereby producing slight flexion to the hip and knee. This results in relaxation of the extensor muscles. While it is contemplated that injection may be performed via the lateral route, the medial route or the anterior route, the lateral route is preferred. After sterile preparation and draping of the skin along the lateral aspect of the patella, the patella is preferably pushed slightly laterally to open the joint. The sift tissues just inferior (posterior) to the lateral patella and its midportion can be anesthetized. For example, a 22 gauge (0.644 mm) needle may be directed anteriorly and cephalad (at about 45 degrees in each direction) until the patella is contacted. Aspiration of joint fluid is then preferably attempted.

The total volume or a partial volume of the diluted composition according to the invention is administered, preferably injected, into the knee joint within a few seconds or minutes. After administration is complete, the injection needle is removed.

Intraarticular administration may be monolateral or bilateral.

Preferably, the patient is a human, preferably from 35 to 85 years of age.

In preferred embodiments a patient is treated who has insufficient pain relief on optimal standard of care.

In preferred embodiments a patient is treated who is unable to be on optimal standard of care due to contraindication or intolerability.

In this regard, optimal standard of care includes one or more of non-pharmacological treatment, oral or topical treatment with NSAIDS, oral treatment with opioids, intraarticular treatment with corticosteroids, and intraarticular treatment with hyaluronic acid.

In preferred embodiments a patient is treated who prior to intraarticular administration has not been locally anesthetized at the site of intraarticular administration.

In other preferred embodiments a patient is treated who prior to intraarticular administration has been locally anesthetized at the site of intraarticular administration, preferably with Ropivacaine, Bupivacaine or Lidocaine.

Preferably, the administered dose unit has a total volume which is smaller than the total volume of the diluted composition.

Preferably, the administered dose unit has a total volume of at least 1.5 ml, more preferably at least 2.0 ml, still more preferably at least 2.5 ml, yet more preferably at least 3.0 ml, even more preferably at least 3.5 ml, most preferably at least 4.0 ml, and in particular at least 4.5 ml.

Preferably, the administered dose unit has a total volume of at most 9.0 ml, preferably at most 8.5 ml, more preferably at most 8.0 ml, still more preferably at most 7.5 ml, yet more preferably at most 7.0 ml, even more preferably at most 6.5 ml, most preferably at most 6.0 ml, and in particular at most 5.5 ml.

Preferably, the administered dose unit has a total volume within the range of 5.0±4.0 ml, preferably 5.0±3.5 ml, more preferably 5.0±3.0 ml, still more preferably 5.0±2.5 ml, yet more preferably 5.0±2.0 ml, even more preferably 5.0±1.5 ml, most preferably 5.0±1.0 ml, and in particular 5.0±0.5 ml.

Preferably, the administered dose unit contains Resiniferatoxin at a dose of at least 10 ng, preferably at least 25 ng, more preferably at least 50 ng, still more preferably at least 75 ng, yet more preferably at least 100 ng, even more preferably at least 125 ng, most preferably at least 150 ng, and in particular at least 175 ng.

Preferably, the administered dose unit contains Resiniferatoxin at a dose of at least 200 ng, preferably at least 225 ng, more preferably at least 250 ng, still more preferably at least 275 ng, yet more preferably at least 300 ng, even more preferably at least 325 ng, most preferably at least 350 ng, and in particular at least 375 ng.

Preferably, the administered dose unit contains Resiniferatoxin at a dose of at most 600 ng, preferably at most 575 ng, more preferably at most 550 ng, still more preferably at most 525 ng, yet more preferably at most 500 ng, even more preferably at most 475 ng, most preferably at most 450 ng, and in particular at most 425 ng.

Preferably, the administered dose unit contains Resiniferatoxin at a dose within the range of 400±200 ng, preferably 400±175 ng, more preferably 400±150 ng, still more preferably 400±125 ng, yet more preferably 400±100 ng, even more preferably 400±75 ng, most preferably 400±50 ng, and in particular 400±25 ng.

Another aspect of the invention relates to a glass vial comprising the liquid pharmaceutical concentrate according to the invention as described above. The glass vial is typically a primary packaging of the concentrate. Every glass vial typically contains medication for a single administration (single dose unit).

Preferably, the glass vial comprises or essentially consists of borosilicate glass.

Preferably, the glass vial comprises or essentially consists of a mixture of SiO₂, B₂O₃, Na₂O, Al₂O₃, and CaO.

Preferably, the glass vial has an inner surface that is coated with a barrier coating layer.

Preferably, the barrier coating layer essentially consists of SiO₂.

Preferably, the barrier coating layer has a thickness within the range of from 100 to 200 nm.

Preferably, the glass vial is resistant against leaching of ions.

Preferably, after filling the empty glass vial with 0.4 M HCl and autoclaving the thus filled glass vial at 121° C. for 1 h, the concentration of leached ions determined by atomic absorption spectroscopy (AAS) is <0.01 μg·ml⁻¹ Na⁺; <0.05 μg·ml⁻¹ Ca²⁺; <0.10 μg·ml⁻¹ B³⁺; <0.30 μg·ml⁻¹ Si⁴⁺; and/or <0.05 μg·ml⁻¹ A³⁺.

Preferably, the glass vial comprises an inert gas.

Preferably, the inert gas comprises or essentially consists of nitrogen, argon, carbon dioxide, nitrous oxide, and mixtures thereof. Preferably, the glass vial comprises a closure system; preferably it is sealed with stoppers and crimped; more preferably it comprises a fluoro-coated bromobutyl stopper with aluminum flip-off seal.

Preferably, the glass vial has a fill capacity of at least 2.5 ml, preferably at least 3.0 ml, more preferably at least 3.5 ml, still more preferably at least 4.0 ml, yet more preferably at least 4.5 ml, even more preferably at least 5.0 ml, most preferably at least 5.5 ml, and in particular at least 6.0 ml.

Preferably, the glass vial has a fill capacity of at least 3.0 ml, preferably at least 4.0 ml, more preferably at least 5.0 ml, still more preferably at least 6.0 ml, yet more preferably at least 7.0 ml, even more preferably at least 8.0 ml, most preferably at least 9.0 ml, and in particular at least 10 ml.

In preferred embodiments, the glass vial containing the liquid pharmaceutical concentrate provides sufficient storage stability and shelf-life after storage at temperatures below 0° C., e.g. −18° C. Preferably, when stored at −18° C. for 6 months, the glass vial containing the liquid pharmaceutical concentrate contains at least 84%, preferably at least 86%, more preferably at least 88%, still more preferably 90%, yet more preferably at least 92%, even more preferably at least 94%, most preferably at least 96%, and in particular at least 98% of the amount of Resiniferatoxin that was contained in the glass vial prior to storage.

In other preferred embodiments, the glass vial containing the liquid pharmaceutical concentrate provides sufficient storage stability and shelf-life after storage at about 5° C. Preferably, when stored at 5° C. for 6 months, the glass vial containing the liquid pharmaceutical concentrate contains at least 84%, preferably at least 86%, more preferably at least 88%, still more preferably 90%, yet more preferably at least 92%, even more preferably at least 94%, most preferably at least 96%, and in particular at least 98% of the amount of Resiniferatoxin that was contained in the glass vial prior to storage.

In further preferred embodiments, the glass vial containing the liquid pharmaceutical concentrate provides sufficient storage stability and shelf-life after storage at room temperature (25° C.). Preferably, when stored at 25° C. for 6 months, the glass vial containing the liquid pharmaceutical concentrate contains at least 84%, preferably at least 86%, more preferably at least 88%, still more preferably 90%, yet more preferably at least 92%, even more preferably at least 94%, most preferably at least 96%, and in particular at least 98% of the amount of Resiniferatoxin that was contained in the glass vial prior to storage.

Stability is preferably determined in accordance with FDA, Guidance for Industry, Drug Stability Guidelines, and Q1A(R2) Stability Testing of New Drug Substances and Products, preferably in the version valid on Jan. 1, 2024.

Another aspect of the invention relates to a kit comprising
a first kit component, which comprises a liquid pharmaceutical concentrate according to the invention as described above, or a glass vial according to the invention as described above; and
a second kit component, which is separate from the first kit component and comprises a dilution liquid according to the invention as described above.

The kit according to the invention is configured such that the second kit component may be mixed with the first kit component thereby obtaining the diluted composition according to the invention as described above. The diluted composition or a partial volume thereof may then be administered as dose unit.

The kit according to the invention is preferably customized to provide upon combining the first kit component and the second kit component with one another a diluted aqueous ethanolic composition for use in the treatment of knee joint pain, especially osteoarthritic knee joint pain, by injection, preferably intraarticular administration, more preferably by intraarticular injection. Preferably, said diluted composition comprises a single unit dose of Resiniferatoxin. The kit for use according to the invention is preferably customized such that after combining the first kit component and the second kit component with one another, a predefined partial volume of the thus obtained diluted aqueous ethanolic composition is used as a dose unit for administration by injection. Preferably, said dose unit
contains Resiniferatoxin at a dose within the range of 400±200 ng, preferably 400±175 ng, more preferably 400±150 ng, still more preferably 400±125 ng, yet more preferably 400±100 ng, even more preferably 400±75 ng, most preferably 400±50 ng, and in particular 400±25 ng;
contains ethanol at a volume concentration within the range of 5.0±4.0 vol.-%; preferably 5.0±3.5 vol.-%, more preferably 5.0±3.0 vol.-%, still more preferably 5.0±2.5 vol.-%, yet more preferably 5.0±2.0 vol.-%, even more preferably 5.0±1.5 vol.-%, most preferably 5.0±1.0 vol.-%, and in particular 5.0±0.5 vol.-%, relative to the total volume of the dose unit; and/or
has a total volume within the range of 5.0±4.0 ml, preferably 5.0±3.5 ml, more preferably 5.0±3.0 ml, still more preferably 5.0±2.5 ml, yet more preferably 5.0±2.0 ml, even more preferably 5.0±1.5 ml, most preferably 5.0±1.0 ml, and in particular 5.0±0.5 ml.

The following examples further illustrate the invention but are not to be construed as limiting its scope.

### Example 1

A stability study was performed with a solution of 1.6 μg Resiniferatoxin ad 1.0 ml ethanol abs. (1.6 μg·ml⁻¹):

| Resiniferatoxin | | |
|---|---|---|
| (Mr 628.71 | | |
| g · mol⁻¹) | mass | mole |

| | | | | |
|---|---|---|---|---|
| in 1.01 | 1.6 | mg | 2.545 | μmol |
| in 1.0 ml | 1.6 | μg | 2.545 | nmol |
| in 420 μl | 672 | ng | 1.069 | nmol |

Aliquots of 420 μl (330 mg) of said solution thus containing 672 ng Resiniferatoxin were filled into glass vials each having a capacity of 10 ml. Prior to filtration and filling, the glass vials had been sparged with nitrogen. The thus filled glass vials were closed with fluoro-coated bromobutyl grey stoppers with aluminum flip-off seals as closure system.

The glass vials were stored under different storage conditions and the content of Resiniferatoxin was determined after various time points.

Two different glass vials were tested, type I uncoated borosilicate glass (I) and type I plus coated borosilicate glass with an inner barrier coating of SiO₂ having a layer thickness of 100-200 nm (I+).

The results are compiled in the table here below:

| | | | 1 | 2 | 3 | 6 | 9 | 12 | 18 |
|---|---|---|---|---|---|---|---|---|---|
| conditions | [%] | t₀ | month | months | months | months | months | months | months |
| −20° C. | I | 96.5 | 90.2 | 91.5 | 92.3 | 92.3 | 91.6 | 91.8* | 92.7 |
| | I+ | 98.7 | 87.8 | 95.0 | 91.1 | 93.3 | 91.3 | 92.3 | n.t. |
| 5° C. upright | I | 96.5 | 93.8 | 98.0 | 94.8 | 93.3 | 88.6 | 88.9* | 82.5 |
| | I+ | 98.7 | 92.8 | 91.7 | 95.3 | 90.7 | 93.2 | 94.2 | n.t. |
| 5° C. inverted | I | 96.5 | 97.6 | 97.8 | 98.1 | 97.1 | 96.7 | 92.5* | 93.4 |
| | I+ | 98.7 | 96.8 | 98.6 | 97.6 | 95.9 | 98.7 | 97.1 | n.t. |
| 25° C. | I | 96.5 | 92.4 | 88.7 | 86.7 | 79.5 | n.t. | n.t. | n.t. |
| | I+ | 98.7 | 91.9 | 95.9 | 95.8 | 93.2 | 94.6 | 97.0 | n.t. |

n.t. not tested;
*13.5 months

### Example 2

In order to further evaluate the stability performance of the Resiniferatoxin 1.6 μg ml⁻¹, concentrate ethanolic solution, a stability study in different vials as primary packaging was carried out:
Samples 1: Schott Type I borosilicate glass
Samples 2: Schott Type I plus® borosilicate glass coated with an inner barrier coating of SiO₂ having a layer thickness of 100-200 nm
Samples 3: Schott TopLyo® borosilicate glass with hydrophobic inner coating
Samples 4: Nipro® borosilicate glass without inner coating but with higher hydrolytic resistance
Samples 5: COP cyclic olefin polymer

Again, in accordance with Example 1, aliquots of 420 μl (330 mg) thus containing 672 ng Resiniferatoxin were filled into the vials each having a capacity of 10 ml. The thus filled glass vials were closed with fluoro-coated bromobutyl grey stoppers with aluminum flip-off seals as closure system. The glass vials were stored under different storage conditions and the content of Resiniferatoxin was determined after various time points.

For further in depth assessment of the influence of pre-treatment of the vials on stability of Resiniferatoxin in the concentrate, three different pre-treatments were tested for the vials:
Pretreatment w: Washing and drying;
Pretreatment a: Depyrogenization;
Pretreatment b: Depyrogenization and autoclaving.

Results for pretreatment w are compiled in the table here below:

| | | initial | | 3 weeks | | 8 weeks | |
|---|---|---|---|---|---|---|---|
| | | assay | | assay | | assay | |
| washing and drying | | (mean, n = 5) | RSD | (mean, n = 6) | RSD | (mean, n = 5) | RSD |
| 5° C. inverted | 1w | 105.5% | 0.3% | 95.9% | 1.2% | 97.3% | 0.5% |
| | 2w | 106.7% | 1.1% | 98.5% | 1.3% | 99.1% | 0.4% |
| | 3w | 102.7% | 3.5% | 98.1% | 0.9% | 99.0% | 1.3% |
| | 4w | 104.8% | 0.3% | 97.4% | 0.5% | 98.0% | 0.8% |
| | 5* | 102.3% | 1.6% | 95.6% | 1.4% | 94.1% | 1.3% |
| 5° C. upright | 1w | 105.5% | 0.3% | 97.8% | 1.4% | 98.4% | 1.0% |
| | 2w | 106.7% | 1.1% | 98.7% | 0.3% | 99.1% | 0.7% |
| | 3w | 102.7% | 3.5% | 94.4% | 0.9% | 94.0% | 0.5% |
| | 4w | 104.8% | 0.3% | 98.1% | 0.3% | 98.7% | 1.2% |
| | 5* | 102.3% | 1.6% | 95.7% | 1.8% | 94.8% | 1.2% |
| 25° C./60% rh | 1w | 105.5% | 0.3% | 95.5% | 1.0% | 98.5% | 1.2% |
| | 2w | 106.7% | 1.1% | 99.2% | 1.7% | 100.5% | 0.4% |
| | 3w | 102.7% | 3.5% | 98.1% | 0.6% | 98.4% | 1.1% |
| | 4w | 104.8% | 0.3% | 96.6% | 2.5% | 94.4% | 3.7% |
| | 5* | 102.3% | 1.6% | 96.0% | 3.3% | 95.3% | 2.7% |

*as is

| | | 4 months | | 13.5 months | |
|---|---|---|---|---|---|
| | | assay | | assay | |
| washing and drying | | (mean, n = 5) | RSD | (mean, n = 5) | RSD |
| 5° C. inverted | 1w | 93.0% | 0.8% | 97.7% | 0.4% |
| | 2w | 97.0% | 1.8% | 99.2% | 1.5% |
| | 3w | 93.0% | 6.7% | 98.3% | 0.8% |
| | 4w | 94.6% | 1.4% | 89.9% | 1.8% |
| | 5* | 94.8% | 2.2% | 98.7% | 1.1% |
| 5° C. upright | 1w | 95.2% | 0.6% | 99.1% | 1.3% |
| | 2w | 99.1% | 1.3% | 101.0% | 1.1% |
| | 3w | 91.3% | 1.5% | 96.6% | 4.1% |
| | 4w | 98.5% | 1.0% | 97.5% | 1.3% |
| | 5* | 95.3% | 1.1% | 98.3% | 0.9% |
| 25° C./60% rh | 1w | 98.8% | 1.0% | 135.7% | 12.0% |
| | 2w | 105.6% | 1.2% | 151.9% | 6.6% |
| | 3w | 98.0% | 1.0% | 112.6% | 6.7% |
| | 4w | 102.7% | 3.1% | 157.8% | 5.7% |
| | 5* | 96.4% | 1.2% | 89.8% | 1.2% |

*as is

Results for pretreatment a are compiled in the table here below:

| | | initial | | 3 weeks | | 8 weeks | |
|---|---|---|---|---|---|---|---|
| | | assay | | assay | | assay | |
| depyrogenization | | (mean, n = 5) | RSD | (mean, n = 6) | RSD | (mean, n = 5) | RSD |
| 5° C. inverted | 1a | 106.5% | 0.6% | 98.3% | 0.4% | 98.9% | 0.9% |
| | 2a | 106.5% | 0.6% | 99.0% | 0.7% | 99.5% | 0.6% |
| | 3a | 100.9% | 0.4% | 98.1% | 1.4% | 99.1% | 0.4% |
| | 4a | 105.7% | 0.7% | 97.8% | 0.3% | 99.2% | 0.6% |
| 5° C. upright | 1a | 106.5% | 0.6% | 98.7% | 0.4% | 99.4% | 1.1% |
| | 2a | 106.5% | 0.6% | 99.0% | 0.6% | 98.4% | 3.1% |
| | 3a | 100.9% | 0.4% | 98.0% | 1.0% | 99.6% | 0.4% |
| | 4a | 105.7% | 0.7% | 98.1% | 0.3% | 99.2% | 0.4% |
| 25° C./60% rh | 1a | 106.5% | 0.6% | 99.0% | 0.6% | 99.8% | 0.7% |
| | 2a | 106.5% | 0.6% | 99.6% | 0.2% | 100.3% | 0.4% |
| | 3a | 100.9% | 0.4% | 96.9% | 1.9% | 99.0% | 1.0% |
| | 4a | 105.7% | 0.7% | 98.9% | 0.5% | 100.7% | 0.2% |

| | | 4 months | | 13.5 months | |
|---|---|---|---|---|---|
| | | assay | | assay | |
| depyrogenization | | (mean, n = 5) | RSD | (mean, n = 5) | RSD |
| 5° C. inverted | 1a | 95.3% | 0.8% | 99.9% | 1.4% |
| | 2a | 98.5% | 0.4% | 101.3% | 0.7% |
| | 3a | 96.8% | 0.3% | 100.3% | 0.8% |
| | 4a | 98.6% | 0.7% | 99.0% | 1.6% |
| 5° C. upright | 1a | 96.7% | 0.5% | 101.4% | 0.7% |
| | 2a | 99.0% | 0.3% | 101.2% | 0.7% |
| | 3a | 96.3% | 2.3% | 101.0% | 3.0% |
| | 4a | 97.3% | 3.7% | 99.3% | 1.0% |
| 25° C./60% rh | 1a | 102.6% | 0.5% | 121.6% | 5.4% |
| | 2a | 103.7% | 2.3% | 141.8% | 3.2% |
| | 3a | 101.7% | 2.6% | 143.1% | 15.9% |
| | 4a | 103.2% | 1.0% | 141.1% | 5.9% |

Results for pretreatment b are compiled in the table here below:

| | | initial | | 3 weeks | | 8 weeks | |
|---|---|---|---|---|---|---|---|
| depyrogenization | | assay | | assay | | assay | |
| and autoclaving | | (mean, n = 5) | RSD | (mean, n = 6) | RSD | (mean, n = 5) | RSD |
| 5° C. inverted | 1b | 105.9% | 0.6% | 96.9% | 0.7% | 97.1% | 1.2% |
| | 2b | 106.1% | 0.7% | 99.2% | 0.4% | 99.2% | 0.7% |
| | 3b | 101.7% | 0.3% | 97.4% | 1.1% | 98.3% | 1.0% |
| | 4b | 105.8% | 0.8% | 96.6% | 0.7% | 96.8% | 1.1% |
| 5° C. upright | 1b | 105.9% | 0.6% | 98.5% | 0.6% | 99.7% | 0.2% |
| | 2b | 106.1% | 0.7% | 98.9% | 0.6% | 100.1% | 1.3% |
| | 3b | 101.7% | 0.3% | 98.4% | 0.8% | 99.9% | 0.3% |
| | 4b | 105.8% | 0.8% | 98.2% | 1.0% | 99.7% | 0.4% |
| 25° C./60% rh | 1b | 105.9% | 0.6% | 98.4% | 0.4% | 99.4% | 0.8% |
| | 2b | 106.1% | 0.7% | 98.8% | 1.0% | 100.2% | 0.5% |
| | 3b | 101.7% | 0.3% | 97.1% | 2.3% | 98.8% | 1.5% |
| | 4b | 105.8% | 0.8% | 99.0% | 0.3% | 100.6% | 0.7% |

| | | 4 months | | 13.5 months | |
|---|---|---|---|---|---|
| depyrogenization | | assay | | assay | |
| and autoclaving | | (mean, n = 5) | RSD | (mean, n = 5) | RSD |
| 5° C. inverted | 1b | 95.8% | 0.9% | 97.0% | 0.5% |
| | 2b | 97.5% | 0.8% | 98.7% | 1.5% |
| | 3b | 95.9% | 1.2% | 100.0% | 0.7% |
| | 4b | 96.4% | 0.3% | 92.5% | 2.9% |
| 5° C. upright | 1b | 98.9% | 0.4% | 100.2% | 0.2% |
| | 2b | 99.1% | 6.7% | 101.3% | 0.8% |
| | 3b | 98.2% | 0.7% | 102.3% | 1.0% |
| | 4b | 99.0% | 0.4% | 100.2% | 0.7% |
| 25° C./60% rh | 1b | 98.9% | 1.5% | 102.2% | 1.7% |
| | 2b | 103.4% | 2.0% | 138.0% | 2.4% |
| | 3b | 96.3% | 8.4% | 116.6% | 7.9% |
| | 4b | 102.3% | 1.7% | 122.8% | 4.0% |

Results above 110% are due to leaching.

The experimental data demonstrate that Samples 2, which are made of borosilicate glass coated with an inner barrier coating of SiO₂ having a layer thickness of 100-200 nm (Schott Type I plus®) provide the best storage stability to the ethanolic concentrates of Resiniferatoxin.

### Example 3

The primary objective of this study was to compare the analgesic effects of single intraarticular injections of Resiniferatoxin (100 or 400 ng) compared to placebo, at 3 and 6 months postinjection, in participants with chronic osteoarthritic knee joint pain. Secondary objectives were to evaluate the analgesic effects of Resiniferatoxin versus placebo in terms of a responder analysis (percentage of subjects achieving ≥50% or ≥70% reduction in visual analog scale pain score) and changes in Western Ontario and McMaster Universities Arthritis Index (WOMAC) total and subscale scores (pain, physical function and stiffness). Safety and injection site pain were also evaluated.

The study was conducted as a randomized, double-blind, placebo-controlled, single-dose trial of Resiniferatoxin. Eligible patients were aged 40 to 80 years with radiographic knee osteoarthritis (Kellgren-Lawrence Grade 2-4 in the last 3 years) and a baseline visual analog scale (0-100 mm) pain score ≥40 mm on motion in the target knee, with or without pain medication. 67 subjects were randomized to treatment with either Resiniferatoxin 100 ng (n=24) or 400 ng (n=23), or placebo (n=20), administered intraarticularly into the index knee. Intraarticular Ropivacaine (5 ml, 0.5%) was administered 15 mins before investigational medicinal product. Visual analog scale scores were used to evaluate pain on motion as the average of the last 2 days in a target knee between baseline and at 3 and 6 months post injection. Injection site pain was rated (visual analog scale) from baseline (−0.5 h) up to 3 h post intraarticular injections.

Baseline demographic characteristics and osteoarthritis pain scores were comparable across treatment groups. In the intention-to-treat population, a reduction in the visual analog scale scores for pain on motion in the treated knee after intraarticular injection was observed as early as the first trial visit post-injection (day 8) with the effect lasting until the last trial visit (month 6; FIG. 1). Subjects receiving Resiniferatoxin 100 ng or Resiniferatoxin 400 ng reported a greater pain reduction than subjects receiving placebo. At the 3 months visit, there was a higher mean [SD] absolute reduction of visual analog scale score (baseline corrected) in the Resiniferatoxin 100 ng group (37.43 [19.79]) and the Resiniferatoxin 400 ng group (36.68 [34.16]) than in the placebo group (17.00 [23.09]). At 6 months, the mean (SD) absolute reduction of visual analog scale score was 33.52 (22.89) in the Resiniferatoxin 100 ng group, 41.48 (32.57) in the Resiniferatoxin 400 ng group, and 28.26 (25.02) in the placebo group. Consistent with these data, the Resiniferatoxin 100 ng and Resiniferatoxin 400 ng treatment groups showed a greater reduction in WOMAC total and subscale scores (pain, physical function and stiffness) than the placebo group at both 3 and 6 months.

The incidence of treatment emergent adverse events in the Resiniferatoxin 400 ng group (78.3%) and placebo group (75.0%) were comparable, but lower in the Resiniferatoxin 100 ng group (62.5%). The most commonly reported treatment emergent adverse events were arthralgia, back pain, nasopharyngitis and headache. The majority of treatment emergent adverse events were mild and unrelated to investigational medicinal product or Ropivacaine. Seven severe adverse events were reported in 6 subjects [2 subjects (Resiniferatoxin 100 ng), 3 subjects (Resiniferatoxin 400 ng) 1 subject (placebo)], none of which were considered to be related to the investigational medicinal product or Ropivacaine. No safety concerns were raised based on other evaluated safety parameters. Injection site pain (procedural pain) was highly variable across treatment groups, although generally higher following intraarticular Resiniferatoxin compared to placebo, with peak pain (moderate-severe intensity in some subjects) noted at around 0.5 h post-injection and return to baseline by 3 h.

The data from this exploratory trial indicate that single intraarticular doses of Resiniferatoxin (100 ng or 400 ng) has the potential to mediate clinically-meaningful pain relief in an osteoarthritic knee population. Analgesic onset occurred within one week of administration and was evident for at least 3 months during the follow-up period. Injection site pain was a very common event. Resiniferatoxin was found to have a good safety profile and to have been well tolerated.

FIG. 1 shows the efficacy in terms of visual analog scale pain in motion, change over time, 2-day recall, intention-to-treat population; n=number of subjects with data; VAS=visual analogue scale. Data values plotted as mean±standard error of mean, offset in X for clarity. ▴ Placebo (n=20); ▪ Resiniferatoxin 100 ng (n=24); ● Resiniferatoxin 400 ng (n=23).

1. A liquid pharmaceutical concentrate comprising or essentially consisting of
(i) Resiniferatoxin in an amount of less than 3.20 μg; and
(ii) ethanol.

2. (canceled)

3. The concentrate according to claim 1, wherein the amount of Resiniferatoxin is at most 700 ng.

4. The concentrate according to claim 3, wherein the amount of Resiniferatoxin is within the range of 460±200 ng.

5. (canceled)

6. The concentrate according to claim 1, which contains Resiniferatoxin at a concentration of at least 0.20 μg·ml⁻¹ and at most 7.00 μg·ml⁻¹.

7. (canceled)

8. (canceled)

9. (canceled)

10. (canceled)

11. (canceled)

12. (canceled)

13. The concentrate according to claim 1, which has a total volume of at least 50 μl and at most 800 μl.

14. (canceled)

15. (canceled)

16. (canceled)

17. The concentrate according to claim 1, which essentially consists of Resiniferatoxin and ethanol.

18. A method for treating pain in a patient in need thereof, said method comprising providing a concentrate according to claim 1, diluting the concentrate with a dilution liquid to obtain a diluted composition, and administering the diluted composition to the patient.

19. The method according to claim 18, wherein the pain is associated with knee osteoarthritis.

20. (canceled)

21. (canceled)

22. (canceled)

23. The method according to claim 18, wherein the dilution liquid is aqueous.

24. The method according to claim 23, wherein the dilution liquid contains a surfactant.

25. The method according to claim 24, wherein the surfactant is D-α-tocopherol polyethylene glycol succinate (TPGS) selected from TPGS 200, TPGS 238, TPGS 400, TPGS 600, TPGS 1000, TPGS 2000, TPGS 3400, TPGS 3500, TPGS 4000, TPGS 6000, and mixtures thereof.

26. (canceled)

27. The method according to claim 23, wherein the dilution liquid is buffered.

28. The method according to claim 23, wherein the dilution liquid has a pH value within the range of 8.0±0.8.

29. The method according to claim 23, wherein the dilution liquid contains one or more isotonizing agents.

30. The method according to claim 23, wherein the dilution liquid has a total volume of at least 4.5 ml and at most 12.0 ml.

31. (canceled)

32. (canceled)

33. (canceled)

34. (canceled)

35. (canceled)

36. (canceled)

37. (canceled)

38. The method according to claim 18, wherein the diluted composition or a partial volume thereof is administered as a dose unit.

39. The method according to claim 38, wherein the administered dose unit is administered by intraarticular injection.

40. The method according to claim 39, wherein the administered dose unit has a total volume which is smaller than the total volume of the diluted composition.

41. (canceled)

42. (canceled)

43. (canceled)

44. The method according to claim 38, wherein the administered dose unit contains Resiniferatoxin at a dose of at least 200 ng and at most 600 ng.

45. (canceled)

46. (canceled)

47. A glass vial comprising the concentrate according to claim 1.

48. The glass vial according to claim 47, which comprises or essentially consists of borosilicate glass.

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. A kit comprising
(i) a first kit component, which comprises a liquid pharmaceutical concentrate according to claim 1; and
(ii) a second kit component, which is separate from the first kit component and comprises a dilution liquid.

61. (canceled)

62. (canceled)

63. The kit according to claim 60, wherein said diluted composition comprises a single unit dose of Resiniferatoxin.

64. (canceled)

65. The kit according to claim 64, wherein said pharmaceutical composition contains Resiniferatoxin at a dose within the range of 400±25 ng.

66. (canceled)

67. (canceled)
